(19) United States
(12) Patent Application Publication
(10) Pub. No.: US 2019/0177493 A1
(43) Pub. Date: Jun. 13, 2019

(54) GEL COMPOSITION, METHOD FOR MANUFACTURING THE GEL COMPOSITION, AND METHOD FOR MANUFACTURING AN OPHTHALMIC LENS USING THE GEL COMPOSITION

(71) Applicant: HON HAI PRECISION INDUSTRY CO., LTD., New Taipei (TW)

(72) Inventors: HSIU-WEN CHIEN, New Taipei (TW); JIAN-CHIAO WANG, New Taipei (TW)

(21) Appl. No.: 15/865,281

(22) Filed: Jan. 9, 2018

(30) Foreign Application Priority Data
Dec. 7, 2017 (CN) 201711290623.7

(51) Int. Cl. C08J 3/075; C08J 3/24; C08B 37/16; C07C 49/713; B01J 13/00; G02B 1/04; G02B 1/12

(52) U.S. Cl. CPC C08J 3/075; C08J 3/24; C08B 37/0012; C07C 49/713; B01J 13/0065; G02B 2207/109; G02B 1/041; G02B 1/12; C08J 2300/12; C08J 2305/16; B01J 13/0069

(57) ABSTRACT

A gel composition comprises hydrophilic monomers, a cross-linking agent, an initiator, and astaxanthin-modified cyclodextrin complexes. The astaxanthin-modified cyclodextrin complexes comprise astaxanthin and modified cyclodextrin. The modified cyclodextrin has a chemical structure of

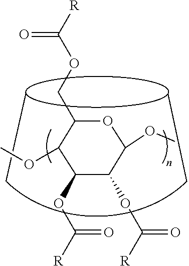

with a hollow cavity, wherein R represents a chemical group having carbon-carbon double bond. The astaxanthin is embedded in the hollow cavity. The disclosure also provides a method for manufacturing a gel composition, and a method for manufacturing an ophthalmic lens using the gel composition.

### FIELD

The subject matter herein generally relates to a gel composition, a method for manufacturing the gel composition, a method for manufacturing an ophthalmic lens.

### BACKGROUND

Contact lenses are commonly worn by users to correct vision, or for cosmetic or therapeutic reasons. However, eyes can suffer from acute red eye or microbial keratitis.

### BRIEF DESCRIPTION OF THE DRAWINGS

Implementations of the present disclosure will now be described, by way of example only, with reference to the attached figures.

FIG. 1 is a flowchart of an exemplary embodiment of a method for manufacturing a gel composition.

FIG. 2 is a flowchart of an exemplary embodiment of a method for manufacturing astaxanthin-modified cyclodextrin complexes in a gel composition.

### DETAILED DESCRIPTION

It will be appreciated that for simplicity and clarity of illustration, where appropriate, reference numerals have been repeated among the different figures to indicate corresponding or analogous elements. In addition, numerous specific details are set forth in order to provide a thorough understanding of the embodiments described herein. However, it will be understood by those of ordinary skill in the art that the embodiments described herein can be practiced without these specific details. In other instances, methods, procedures, and components have not been described in detail so as not to obscure the related relevant feature being described. Also, the description is not to be considered as limiting the scope of the embodiments described herein. The drawings are not necessarily to scale and the proportions of certain parts may be exaggerated to better illustrate details and features of the present disclosure.

The term “comprising,” when utilized, means “including, but not necessarily limited to”; it specifically indicates open-ended inclusion or membership in the so-described combination, group, series, and the like.

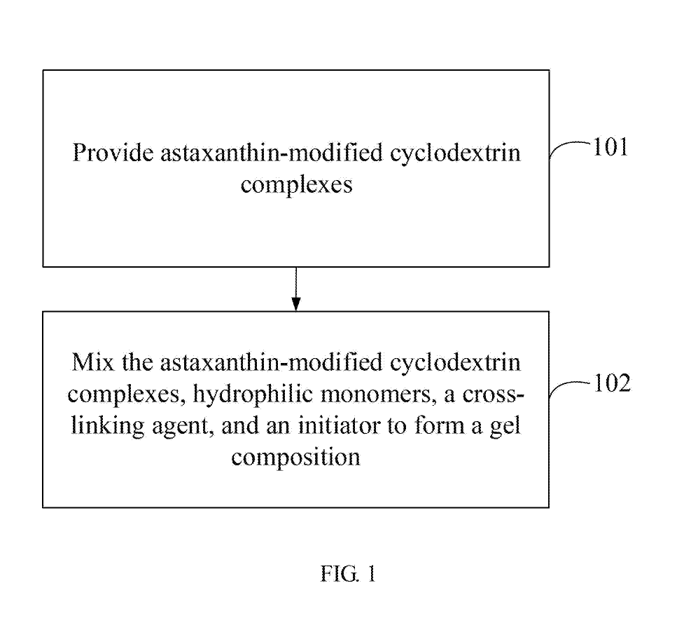

FIG. 1 illustrates a flowchart of a method for manufacturing a gel composition in accordance with an exemplary embodiment. The exemplary method is provided by way of example, as there are a variety of ways to carry out the method. Each block shown in FIG. 1 represents one or more processes, methods or subroutines, carried out in the exemplary method. Furthermore, the illustrated order of blocks is by example only and the order of the blocks can change. Additional blocks may be added or fewer blocks may be utilized, without departing from this disclosure. The exemplary method can begin at block 101.

At block 101, astaxanthin-modified cyclodextrin complexes are provided. The astaxanthin-modified cyclodextrin complexes comprise astaxanthin and modified cyclodextrin. Each modified cyclodextrin has a hollow cavity, and the astaxanthin is embedded in the hollow cavities.

Cyclodextrin is a generic term for a series of cyclic oligosaccharides produced by amylose reacted under cyclodextrin glucosyltransferase produced by bacillus. Cyclodextrin contains 6 to 12 D-glucopyranose units. Based on the results of X-ray crystallography, infrared spectroscopy and nuclear magnetic resonance spectroscopy, each glucopyranose unit constituting a cyclodextrin molecule has a chair or stool configuration. Each glucose unit is bound to a ring with 1, 4-glycosidic bonds. Since glucosidic linkages connecting the glucose units are not free to rotate, the cyclodextrin molecule is not a cylindrical molecule but is slightly conical, i.e., a frustum, and has a hollow cavity. The hydrophilic functional groups, such as hydroxyl groups, are located at exterior of the hollow cavity of the cyclodextrin molecule. Secondary hydroxyl groups are located at a larger open end of the hollow cavity, and primary hydroxyl groups are located at a smaller open end of the hollow cavity, so the outside of the cyclodextrin is hydrophilic. Interior of the hollow cavity is relatively hydrophobic.

The modified cyclodextrin has a chemical structure of

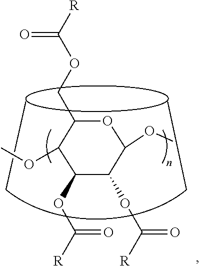

wherein R represents a chemical group having carbon-carbon double bond. A degree of polymerization n is from about 6 to about 12. The astaxanthin has a chemical structure of

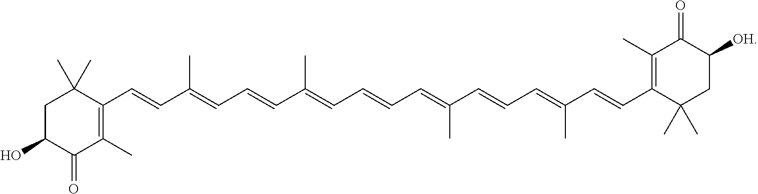 [SMILES: CC1=C(/C=C/C(C)=C/C=C/C(C)=C/C=C/C=C(C)/C=C/C=C(C)/C=C/C2=C(C)C(=O)[C@@H](O)CC2(C)C)C(C)(C)C[C@H](O)C1=O]

In at least one exemplary embodiment, the modified cyclodextrin is methacrylate modified cyclodextrin having a chemical structure of

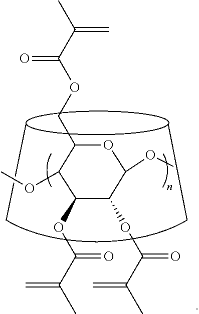

At block 102, a silicone hydrogel composition comprising the astaxanthin-modified cyclodextrin complexes is provided. The astaxanthin-modified cyclodextrin complexes have a mass percentage of about 0.08% to about 15.37% of a total mass of the gel composition.

The silicone hydrogel composition further comprises hydrophilic monomers, a cross-linking agent, and an initiator. The hydrophilic monomers, the cross-linking agent, the initiator, and the astaxanthin-modified cyclodextrin complexes are mixed together. In at least one exemplary embodiment, the hydrophilic monomers have a mass percentage of about 56% to about 99.52% of the total mass of the gel composition. The cross-linking agent has a mass percentage of about 0.03% to about 21.82% of the total mass of the gel composition. The initiator has a mass percentage of about 0.042% to about 18.62% of the total mass of the gel composition.

The hydrophilic monomers may be selected from a group consisting of methacryloxyalkyl siloxanes, 3-methacryloxypropylpentamethyldisiloxane, bis(methacryloxypropyl)tetramethyl-disiloxane, monomethacrylatedpolydimethylsiloxane, mercapto-terminatedpolydimethylsiloxane, N-[tris(trimethylsiloxy)silylpropyl]acrylamide, N-[tris(trimethylsiloxy)silylpropyl]methacrylamide, tris(pentamethyldisiloxyanyl)-3-methacrylatopropylsilane (T2), 3-methacryloxypropyletris(trimethylsiloxy)silane, 2-hydroxyethylmethacrylate (HEMA), methyl methacrylate, hydroxyethyl acrylate, hydroxypropyl acrylate, hydroxypropyl methacrylate (HPMA), trimethylammonium 2-hydroxy propylmethacrylate hydrochloride, dimethylaminoethyl methacrylate (DMAEMA), dimethylaminoethylmethacrylamide, acrylamide, methacrylamide, allyl alcohol, vinylpyridine, glycerol methacrylate, N-(1,1dimethyl-3-oxobutyl)acrylamide, N-vinyl-2-pyrrolidone (NVP), acrylic acid, methacrylic acid, and N,N-dimethyacrylamide (DMA), and any combination thereof.

The initiator may be a photoinitiator or a thermal initiator.

The photoinitiator may be selected from a group consisting of benzoin methyl ether, diethoxyacetophenone, a benzoylphosphine oxide initiator, ethyl 2-dimethylaminobenzoate, 2-isopropylthioxanthone, 1-hydroxycyclohexyl phenyl ketone, Darocur type initiator and Irgacur type initiator, and any combination thereof. In at least one exemplary embodiment, the photoinitiator may be Irgacure-1173. The benzoylphosphine oxide initiator may be selected from a group consisting of 2,4,6-trimethylbenzoyldiphenylophosphine oxide, bis-(2,6-dichlorobenzoyl)-4-N-propylphenylphosphine oxide, and bis-(2,6-dichlorobenzoyl)-4-N-butylphenylphosphine oxide, and any combination thereof.

The thermal initiator may be selected from a group consisting of 2,2′-azobis (2,4-dimethylpentanenitrile), 2,2′-azobis(2-methylpropanenitrile), 2,2′-azobis (2-methylbutanenitrile), azobisisobutyronite (AIBN), and peroxide, and any combination thereof. The peroxide can be benzoyl peroxide.

The cross-linking agent may be selected from a group consisting of ethylene glycol dimethacrylate (EGDMA), trimethylolpropane trimethacrylate (TMPTMA), tri(ethylene glycol) dimethacrylate (TEGDMA), tri(ethylene glycol) divinyl ether (TEGDVE), and trimethylene glycol dimethacrylate, and any combination thereof.

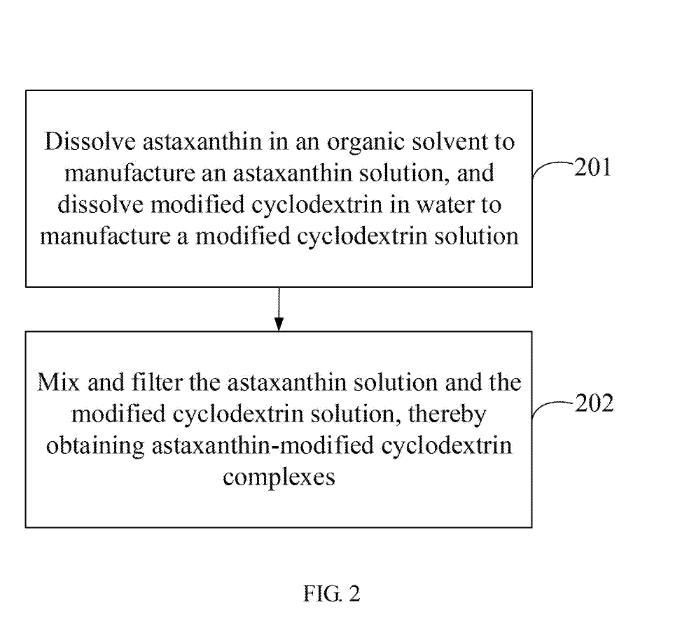

FIG. 2 illustrates a flowchart of a method for manufacturing the astaxanthin-modified cyclodextrin complexes. The exemplary method can begin at block 201.

At block 201, astaxanthin is dissolved in an organic solvent to manufacture an astaxanthin solution, and modified cyclodextrin is dissolved in water to manufacture a modified cyclodextrin solution.

In at least one exemplary embodiment, the astaxanthin solution has a concentration of about 0.0001 mol/L to about 0.1 mol/L. The modified cyclodextrin solution has a concentration of about 0.0001 mol/L to about 0.1 mol/L.

In at least one exemplary embodiment, the organic solvent may be selected from a group consisting of tetrahydrofuran, ethanol, and acetone, and any combination thereof.

In another exemplary embodiment, the solvent may be selected from a group consisting of tripropylene glycol methyl ether, dipropylene glycol methyl ether, ethylene glycol n-butyl ether, diethylene glycol n-butyl ether, diethylene glycol methyl ether, ethylene glycol phenyl ether, propylene glycol methyl ether, propylene glycol methyl ether acetate, dipropylene glycol methyl ether acetate, propylene glycol n-propyl ether, dipropylene glycol n-propyl ether, tripropylene glycol n-butyl ether, propylene glycol n-butyl ether, dipropylene glycol n-butyl ether, tripropylene glycol n-butyl ether, propylene glycol phenyl ether dipropylene glycol dimethyl ether, polyethylene glycols, polypropylene glycols, ethyl acetate, butyl acetate, amyl acetate, methyl lactate, ethyl lactate, i-propyl lactate, methylene chloride, 2-butanol, 2-propanol, menthol, cyclohexanol, cyclopentanol and exonorborneol, 2-pentanol, 3-pentanol, 2-hexanol, 3-hexanol, 3-methyl-2-butanol, 2-heptanol, 2-octanol, 2-nonanol, 2-decanol, 3-octanol, norborneol, tert-butanol, tert-amyl, alcohol, 2-methyl-2-pentanol, 2,3-dimethyl-2-butanol, 3-methyl-3-pentanol, 1-methylcyclohexanol, 2-methyl-2-hexanol, 3,7-dimethyl-3-octanol, 1-chloro-2-methyl-2-propanol, 2-methyl-2-heptanol, 2-methyl-2-octanol, 2-2-methyl-2-nonanol, 2-methyl-2-decanol, 3-methyl-3-hexanol, 3-methyl-3-heptanol, 4-methyl-4-heptanol, 3-methyl-3-octanol, 4-methyl-4-octanol, 3-methyl-3-nonanol, 4-methyl-4-nonanol, 3-methyl-3-octanol, 3-ethyl-3-hexanol, 3-methyl-3-heptanol, 4-ethyl-4-heptanol, 4-propyl-4-heptanol, 4-isopropyl-4-heptanol, 2,4-dimethyl-2-pentanol, 1-methylcyclopentanol, 1-ethylcyclopentanol, 1-ethylcyclopentanol, 3-hydroxy-3-methyl-1-butene, 4-hydroxy-4-methyl-1-cyclopentanol, 2-phenyl-2-propanol, 2-methoxy-2-methyl-2-propanol 2,3,4-trimethyl-3-pentanol, 3,7-dimethyl-3-octanol, 2-phenyl-2-butanol, 2-methyl-1-phenyl-2-propanol and 3-ethyl-3-pentanol, 1-ethoxy-2-propanol, 1-methyl-2-propanol, t-amyl alcohol, isopropanol, 1-methyl-2-pyrrolidone, N,N-dimethylpropionamide, dimethyl formamide, dimethyl acetamide, dimethyl propionamide, and N-methyl pyrrolidinone, and any combination thereof.

At block 202, astaxanthin-modified cyclodextrin complexes are obtained by mixing and filtering the astaxanthin solution and the modified cyclodextrin solution. Because interior of the hollow cavities of the modified cyclodextrin and the astaxanthin have relative hydrophobicity, the astaxanthin is embedded in the hollow cavities of the modified cyclodextrin when the astaxanthin solution and the modified cyclodextrin solution are mixed, thereby forming the astaxanthin-modified cyclodextrin complexes. The exterior of the cyclodextrin has hydrophilicity, so that the astaxanthin-modified cyclodextrin complexes can dissolve in the gel composition without damaging a structure of the astaxanthin. The astaxanthin in the ophthalmic lens has antioxidant properties and can reduce inflammation in the eyes. Astaxanthin in the ophthalmic lens also resists strong light.

In at least one exemplary embodiment, the astaxanthin solution and the modified cyclodextrin solution are mixed 1:1 in a molar ratio.

Depending on the embodiment, certain of the steps of methods described may be removed, others may be added, and the sequence of steps may be altered. It is also to be understood that the description and the claims drawn to a method may include some indication in reference to certain steps. However, the indication used is only to be viewed for identification purposes and not as a suggestion as to an order for the steps.

An exemplary embodiment of a gel composition comprises hydrophilic monomers, a cross-linking agent, an initiator, and astaxanthin-modified cyclodextrin complexes. The astaxanthin-modified cyclodextrin complexes comprise astaxanthin and modified cyclodextrin. Each modified cyclodextrin has a hollow cavity, and the astaxanthin embeds in these hollow cavities.

The modified cyclodextrin has a chemical structure of

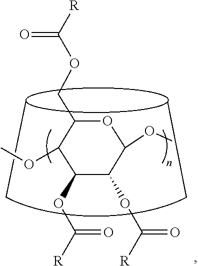

wherein R represents a chemical group having carbon-carbon double bond. A degree of polymerization n is from about 6 to about 12. The astaxanthin has a chemical structure of

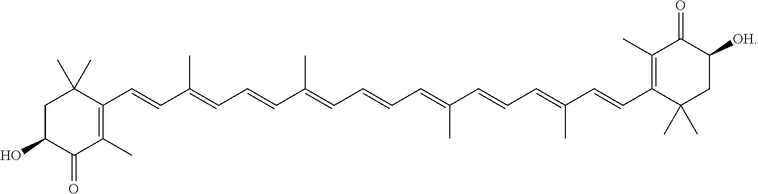 [SMILES: CC1=C(/C=C/C(C)=C/C=C/C(C)=C/C=C/C=C(C)/C=C/C=C(C)/C=C/C2=C(C)C(=O)[C@@H](O)CC2(C)C)C(C)(C)C[C@H](O)C1=O]

In at least one exemplary embodiment, the modified cyclodextrin has a chemical structure of

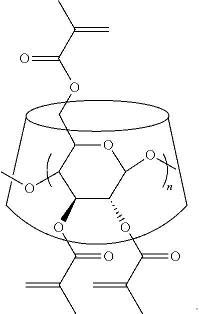

In at least one exemplary embodiment, the astaxanthin-modified cyclodextrin complexes have a mass percentage of about 0.08% to about 15.37% of a total mass of the gel composition. The hydrophilic monomers have a mass percentage of about 56% to about 99.52% of the total mass of the gel composition. The cross-linking agent has a mass percentage of about 0.03% to about 21.82% of the total mass of the gel composition. The initiator has a mass percentage of about 0.042% to about 18.62% of the total mass of the gel composition.

A method for manufacturing an ophthalmic lens comprises a step of feeding the gel composition into a mold and exposing to ultraviolet radiation or heating, so the astaxanthin-modified cyclodextrin complexes, the hydrophilic monomers, the cross-linking agent, and the initiator in the gel composition undergo a polymerization reaction and form an ophthalmic lens. The astaxanthin-modified cyclodextrin complexes react with the hydrophilic monomers by the carbon-carbon double bonds. In at least one exemplary embodiment, the gel composition is exposed to ultraviolet radiation for about 5 min to about 30 min. The gel composition is heated at about 60 degrees Celsius to about 90 degrees Celsius for about 0.5 h to about 5 h.

### Example 1

Astaxanthin is dissolved in tetrahydrofuran to manufacture an astaxanthin solution having concentration of about 0.01 mol/L. Methacrylate modified cyclodextrin is dissolved in water to manufacture a methacrylate modified cyclodextrin solution having concentration of about 0.01 mol/L.

Astaxanthin-modified cyclodextrin complexes are obtained by mixing and filtering the astaxanthin solution and the modified cyclodextrin solution in a ratio of 1:1 by volume.

A gel composition is formed by mixing the astaxanthin-modified cyclodextrin complexes, 2-hydroxyethylmethacrylate, ethylene glycol dimethacrylate, and azobisisobutyronite. The astaxanthin-modified cyclodextrin complexes have a mass percentage of about 0.97% of a total mass of the gel composition. 2-hydroxyethylmethacrylate has a mass percentage of about 98.25% of the total mass of the gel composition. Ethylene glycol dimethacrylate has a mass percentage of about 0.52% of the total mass of the gel composition. Azobisisobutyronite has a mass percentage of about 0.26% of the total mass of the gel composition.

The gel composition is fed into a mold, and heated at about 80 degrees Celsius for about 5 h to form an ophthalmic lens.

### Example 2

Astaxanthin is dissolved in tetrahydrofuran to manufacture an astaxanthin solution having concentration of about 0.01 mol/L. Methacrylate modified cyclodextrin is dissolved in water to manufacture a methacrylate modified cyclodextrin solution having concentration of about 0.01 mol/L.

Astaxanthin-modified cyclodextrin complexes are obtained by mixing and filtering the astaxanthin solution and the modified cyclodextrin solution in a ratio of 1:1 by volume.

A gel composition is formed by mixing the astaxanthin-modified cyclodextrin complexes, 2-hydroxyethylmethacrylate, ethylene glycol dimethacrylate, and azobisisobutyronite. The astaxanthin-modified cyclodextrin complexes have a mass percentage of about 1.67% of a total mass of the gel composition. 2-hydroxyethylmethacrylate has a mass percentage of about 97.79% of the total mass of the gel composition. Ethylene glycol dimethacrylate has a mass percentage of about 0.08% of the total mass of the gel composition. Azobisisobutyronite has a mass percentage of about 0.46% of the total mass of the gel composition.

The gel composition is fed into a mold, and heated at about 80 degrees Celsius for about 5 h to form an ophthalmic lens.

### Example 3

Astaxanthin is dissolved in tetrahydrofuran to manufacture an astaxanthin solution having concentration of about 0.02 mol/L. Methacrylate modified cyclodextrin is dissolved in water to manufacture a methacrylate modified cyclodextrin solution having concentration of about 0.01 mol/L.

Astaxanthin-modified cyclodextrin complexes are obtained by mixing and filtering the astaxanthin solution and the modified cyclodextrin solution in a ratio of 1:2 by volume.

A gel composition is formed by mixing the astaxanthin-modified cyclodextrin complexes, 2-hydroxyethylmethacrylate, methyl methacrylate, ethylene glycol dimethacrylate, and azobisisobutyronite. The astaxanthin-modified cyclodextrin complexes have a mass percentage of about 0.56% of a total mass of the gel composition. 2-hydroxyethylmethacrylate has a mass percentage of about 98.25% of the total mass of the gel composition. Methyl methacrylate has a mass percentage of about 0.45% of the total mass of the gel composition. Ethylene glycol dimethacrylate has a mass percentage of about 0.38% of the total mass of the gel composition. Azobisisobutyronite has a mass percentage of about 0.36% of the total mass of the gel composition.

The gel composition is fed into a mold, and heated at about 80 degrees Celsius for about 5 h to form an ophthalmic lens.

It is to be understood, even though information and advantages of the present embodiments have been set forth in the foregoing description, together with details of the structures and functions of the present embodiments, the disclosure is illustrative only; changes may be made in detail, especially in matters of shape, size, and arrangement of parts within the principles of the present embodiments to the full extent indicated by the plain meaning of the terms in which the appended claims are expressed.

What is claimed is:

1. A method for manufacturing a gel composition comprising:
providing astaxanthin-modified cyclodextrin complexes; and
mixing the astaxanthin-modified cyclodextrin complexes, hydrophilic monomers, a cross-linking agent, and an initiator to form the gel composition;
wherein the astaxanthin-modified cyclodextrin complexes comprise astaxanthin and modified cyclodextrin, the modified cyclodextrin has a chemical structure of 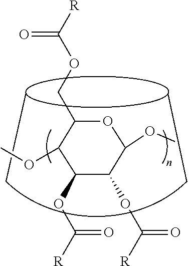
with a hollow cavity, R represents a chemical group having carbon-carbon double bond, the astaxanthin is embedded in the hollow cavity.

2. The method for manufacturing the gel composition of claim 1, wherein the modified cyclodextrin has a chemical structure of

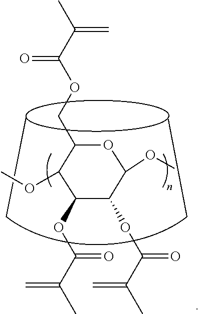

3. The method for manufacturing the gel composition of claim 1, wherein the astaxanthin-modified cyclodextrin complexes have a mass percentage of about 0.08% to about 15.37% of a total mass of the gel composition.

4. The method for manufacturing the gel composition of claim 3, wherein the hydrophilic monomers have a mass percentage of about 56% to about 99.52% of the total mass of the gel composition, the cross-linking agent has a mass percentage of about 0.03% to about 21.82% of the total mass of the gel composition, the initiator has a mass percentage of about 0.042% to about 18.62% of the total mass of the gel composition.

5. The method for manufacturing the gel composition of claim 1, wherein providing the astaxanthin-modified cyclodextrin complexes further comprises:
dissolving astaxanthin in an organic solvent to manufacture an astaxanthin solution, and dissolving modified cyclodextrin in water to manufacture a modified cyclodextrin solution; and
mixing and filtering the astaxanthin solution and the modified cyclodextrin solution, thereby obtaining astaxanthin-modified cyclodextrin complexes.

6. The method for manufacturing the gel composition of claim 5, wherein the astaxanthin solution has a concentration of about 0.0001 mol/L to about 0.1 mol/L, the modified cyclodextrin solution has a concentration of about 0.0001 mol/L to about 0.1 mol/L.

7. The method for manufacturing the gel composition of claim 5, wherein the astaxanthin solution and the modified cyclodextrin solution are mixed 1:1 in a molar ratio.

8. A gel composition comprising:
hydrophilic monomers;
a cross-linking agent;
an initiator; and
astaxanthin-modified cyclodextrin complexes;
wherein the astaxanthin-modified cyclodextrin complexes comprise astaxanthin and modified cyclodextrin, the modified cyclodextrin has a chemical structure of 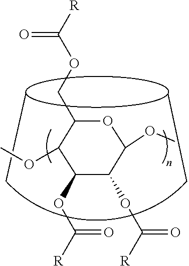
with a hollow cavity, R represents a chemical group having carbon-carbon double bond, the astaxanthin is embedded in the hollow cavity.

9. The gel composition of claim 8, wherein the modified cyclodextrin has a chemical structure of

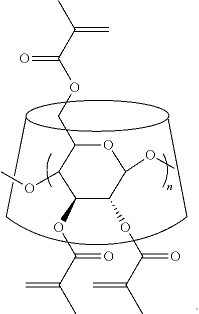

10. The gel composition of claim 8, wherein the astaxanthin-modified cyclodextrin complexes have a mass percentage of about 0.08% to about 15.37% of a total mass of the gel composition.

11. The gel composition of claim 10, wherein the hydrophilic monomers have a mass percentage of about 56% to about 99.52% of the total mass of the gel composition, the cross-linking agent has a mass percentage of about 0.03% to about 21.82% of the total mass of the gel composition, the initiator has a mass percentage of about 0.042% to about 18.62% of the total mass of the gel composition.

12. A method for manufacturing an ophthalmic lens comprising:
providing astaxanthin-modified cyclodextrin complexes;
mixing the astaxanthin-modified cyclodextrin complexes, hydrophilic monomers, a cross-linking agent, and an initiator to form the gel composition, wherein the astaxanthin-modified cyclodextrin complexes comprise astaxanthin and modified cyclodextrin, the modified cyclodextrin has a chemical structure of 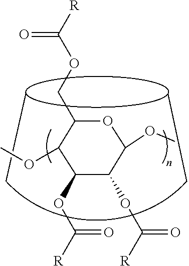
with a hollow cavity, R represents a chemical group having carbon-carbon double bond, the astaxanthin is embedded in the hollow cavity; and
feeding the gel composition into a mold, and exposing the gel composition to ultraviolet radiation or heating the gel composition, to cause the astaxanthin-modified cyclodextrin complexes, the hydrophilic monomers, the cross-linking agent, and the initiator in the gel composition undergo a polymerization reaction, thereby forming the ophthalmic lens.

13. The method for manufacturing the ophthalmic lens of claim 12, wherein the modified cyclodextrin has a chemical structure of

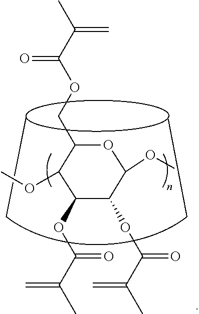

14. The method for manufacturing the ophthalmic lens of claim 12, wherein the astaxanthin-modified cyclodextrin complexes have a mass percentage of about 0.08% to about 15.37% of a total mass of the gel composition.

15. The method for manufacturing the ophthalmic lens of claim 14, wherein the hydrophilic monomers have a mass percentage of about 56% to about 99.52% of the total mass of the gel composition, the cross-linking agent has a mass percentage of about 0.03% to about 21.82% of the total mass of the gel composition, the initiator has a mass percentage of about 0.042% to about 18.62% of the total mass of the gel composition.

16. The method for manufacturing the ophthalmic lens of claim 12, wherein providing the astaxanthin-modified cyclodextrin complexes further comprises:
dissolving astaxanthin in an organic solvent to manufacture an astaxanthin solution, and dissolving modified cyclodextrin in water to manufacture a modified cyclodextrin solution; and
mixing and filtering the astaxanthin solution and the modified cyclodextrin solution, thereby obtaining astaxanthin-modified cyclodextrin complexes.

17. The method for manufacturing the ophthalmic lens of claim 16, wherein the astaxanthin solution has a concentration of about 0.0001 mol/L to about 0.1 mol/L, the modified cyclodextrin solution has a concentration of about 0.0001 mol/L to about 0.1 mol/L.

18. The method for manufacturing the ophthalmic lens of claim 16, wherein the astaxanthin solution and the modified cyclodextrin solution are mixed 1:1 in a molar ratio.

19. The method for manufacturing the ophthalmic lens of claim 12, wherein the gel composition is exposed to ultraviolet radiation for about 5 min to about 30 min, or the gel composition is heated at about 60 degrees Celsius to about 90 degrees Celsius for about 0.5 h to about 5 h.
